(19) United States
(12) Patent Application Publication
(10) Pub. No.: US 2024/0262962 A1
(43) Pub. Date: Aug. 8, 2024

(54) POLYAMIDES AND CORRESPONDING POLYMER COMPOSITIONS AND ARTICLES

(71) Applicant: SOLVAY SPECIALTY POLYMERS USA, LLC, Alpharetta, GA (US)

(72) Inventors: Arthur Bertrand, Villette de Vienne (FR); Stéphane Jeol, Saint-Genis-Laval (FR); Joel Flores, Alpharetta, GA (US)

(73) Assignee: SOLVAY SPECIALTY POLYMERS USA, LLC, Alpharetta, GA (US)

(21) Appl. No.: 18/560,658

(22) PCT Filed: May 11, 2022
(86) PCT No.: PCT/EP2022/062847

§ 371 Date: Nov. 13, 2023

(30) Foreign Application Priority Data
May 11, 2021 (EP) 21305605.4

(51) Int. Cl. C08G 69/26; C08L 77/06

(52) U.S. Cl. CPC C08G 69/265; C08L 77/06

(57) ABSTRACT

Described herein are amorphous and transparent aliphatic polyamides (PA) formed from the polycondensation of monomers in a reaction mixture (RM) including a dicarboxylic acid component (DC) and a diamine component (DA). The dicarboxylic acid component (DC) includes 25 mol % to 80 mol % of 1,4-cyclohexane dicarboxylic acid (“1,4-CHDA”) and 20 mol % to 75 mol % of a linear, aliphatic dicarboxylic acid selected from azelaic acid, sebacic acid or a combination thereof. The diamine component (DA) includes a cycloaliphatic diamine selected from the group consisting of isophoronediamine (“IPDA”); 4,4′-methyl-enebis(2-methylcyclohexylamine) (“MACM”); 4,4′-methylene-bis-cyclohexane (“PACM”); 1,3-bis(aminomethyl)cyclohexane (“1,3-BAC”), bis(aminomethyl)norborane (“BAMN”) and any combination of two or more thereof. It was surprisingly found that the polyamides (PA), while being bio-based (at least in part due to the linear aliphatic dicarboxylic acid), had excellent optical clarity, improved UV stability and desirably high Tg.

The present application claims the priority of European patent application EP 21305605.4 filed on 11 May 2021, the content of which being entirely incorporated herein by reference for all purposes. In case of any incoherency between the present application and the PCT application that would affect the clarity of a term or expression, it should be made reference to the present application only.

### FIELD OF THE INVENTION

The invention relates to a polyamide including recurring units formed from azelaic acid or sebacic acid and having high glass transition temperatures and bio-content. The invention further relates to polymer compositions and articles incorporating the polyamide.

### BACKGROUND OF THE INVENTION

Given the wide proliferation of polyamides in many application settings, there is an ever growing industry demand to develop polyamides from renewable resources. While there have been advances in developing such polyamides based upon, for example, dodecanoic acid, such polyamides have relatively low Tg and, therefore, their suitability for high heat applications is limited.

CN 107383369 discloses a polyamide comprising the recurring units of an aromatic acid (isophthalic acid). There is no mention of the Tg of the polyamide. JP 2003/261766 discloses a polyamide comprising the recurring units of an aromatic acid (isophthalic acid). The Tg of the polyamide is not within the claimed range. DE 1520924 does not disclose a polyamide as claimed. U.S. Pat. No. 2,965,616 does not disclose an amorphous polyamide.

### Technical Problem

There is a need for a polyamide of high Tg which exhibits a good combination of optical clarity and UV stability and which can be biobased. The polyamide of the invention aims at solving this technical problem.

### BRIEF DESCRIPTION OF THE INVENTION

The invention is set out in the appended set of claims. An object of the invention is thus a polyamide as defined in any one of claims 1-24. The polyamide (PA) or the amorphous, aliphatic polyamide (PA) comprises the recurring units R_{PA1} and R_{PA2} which are represented by the following formulae, respectively,

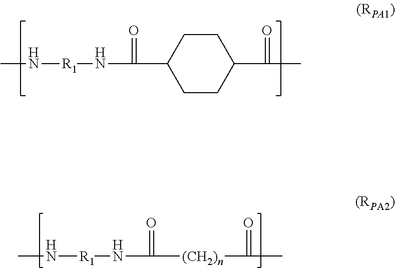

with the following proportions:
from 25.0 to 80.0 mol. % of R_{PA1};
from 20.0 to 75.0 mol. % of R_{PA2};
where:
R₁ is a divalent radical of a diamine selected from the group consisting of isophoronediamine (“IPDA”); 4,4′-methylenebis(2-methylcyclohexylamine) (“MACM”); 4,4′-methylene-bis-cyclohexylamine (“PACM”); 1,3-bis(aminomethyl)cyclohexane (“1,3-BAC”), bis(aminomethyl)norbornane (“BAMN”), 1,2-cyclohexane diamine, 1,3-cyclohexane diamine, 1,4-cyclohexane diamine, 4-methylcyclohexane-1,3-diamine, 2-methylcyclohexane-1,3-diamine, 1,8-diamino-p-menthane, 2,2-bis(4-aminocyclohexyl)propane (“PACP”), 4,4′-methanediylbis(2,6-dimethylcyclohexanamine) and any combination of two or more thereof, the diamine being more particularly selected from the group consisting of IPDA, MACM, PACM, 1,3-BAC, BAMN and any combination of two or more thereof, and
n is 7 or 8;
and wherein the polyamide (PA) has a glass transition temperature (“Tg”) of from 160° C. to 260° C., preferably from 160° C. to 220° C.

Another object of the invention is a composition as defined in any one of claims 25-29. Another object of the invention is an article as defined in any one of claims 30-34. More precisions and details about these objects are now provided below.

### SUMMARY OF INVENTION

In a first aspect, the invention is directed to a amorphous, aliphatic polyamide (PA) including recurring units formed from the polycondensation of a reaction mixture (RM) including: a dicarboxylic acid component (DC) and a diamine component (DA). The dicarboxylic acid component (DC) comprises 25 mol % to 80 mol % of 1,4-cyclohexane dicarboxylic acid (“1,4-CHDA”) and 20 mol % to 75 mol % of a linear, aliphatic dicarboxylic acid selected from the group consisting of azelaic acid (HOOC—(CH₂)₇—COOH), sebacic acid (HOOC—(CH₂)₈—COOH) and a combination thereof, wherein mol % is relative to the total moles of dicarboxylic acids in the dicarboxylic acid component (DA). The diamine component (DA) comprises a cycloaliphatic diamine selected from the group consisting of isophoronediamine (“IPDA”); 4,4′-methylenebis(2-methylcyclohexylamine) (“MACM”); 4,4′-methylene-bis-cyclohexylamine (“PACM”); 1,3-bis(aminomethyl)cyclohexane (“1,3-BAC”), bis(aminomethyl)norbornane (“BAMN”), 1,2-cyclohexane diamine, 1,3-cyclohexane diamine, 1,4-cyclohexane diamine, 4-methylcyclohexane-1,3-diamine, 2-methylcyclohexane-1,3-diamine, 1,8-diamino-p-menthane, 2,2-bis(4-aminocyclohexyl)propane (“PACP”), 4,4′-methanediylbis(2,6-dimethylcyclohexanamine) and any combination of two or more thereof, wherein mol % is relative to the total moles of diamines in the diamine component (DA). The polyamide (PA) has a glass transition temperature (“Tg”) of from 160° C. to 260° C., preferably from 160° C. to 220° C., as measured according to ASTM D3418. The reaction mixture (RM) is free of tertiary amines, lactams and amino acids. Additionally, if the cycloaliphatic dicarboxylic acid is PACM, the PACM comprises a trans-trans ratio of no more than 30%. In some embodiments, the polyamide (PA) is transparent.

In some embodiments, the cycloaliphatic diamine is selected from the group consisting of IPDA, MACM, PACM, 1,3-BAC BAMN and any combination of two or more thereof. In some embodiments, the dicarboxylic acid component (DC) consists essentially of the 1,4-CHDA and linear carboxylic acid. In some embodiments, the diamine component (DA) consists essentially of the cycloaliphatic diamine. In some embodiments, the concentration of the cycloaliphatic diamine is at least 80 mol %. In some embodiments, the polyamide (PA) has a biobased carbon content of at least 20%.

In another aspect, the invention is directed to a polymer composition (C) comprising the polyamide (PA) and a component selected from the group consisting of reinforcing agents, tougheners, plasticizers, colorants, pigments, antistatic agents, dyes, lubricants, thermal stabilizers, light stabilizers, flame retardants, nucleating agents, antioxidants, processing aids and any combination of two or more thereof.

In some embodiments, the polymer composition (C) comprises a reinforcing agent selected from the group consisting of glass fiber and carbon fiber.

In some embodiments, the polymer composition (C) includes a halogen free flame retardant.

In another aspect, the invention is directed to an article comprising the polyamide (PA) or the polymer composition (C), wherein the article is selected from the group consisting of sutures, hemodialysis membranes, vascular catheters, scaffolds, catheter balloons, wound dressings, breathing masks, and medical tubing.

In another aspect, the invention is directed to an article comprising the polyamide (PA) or the polymer composition (C), wherein the article is either a filament or a powder.

In another aspect, the invention is directed to an article comprising the polyamide (PA) or the polymer composition (C), wherein the article is mobile electronic device component.

In another aspect, the invention is directed to an article comprising the polyamide (PA) or the polymer composition (C), wherein the article is an automotive component.

In another aspect, the invention is directed to an article comprising the polyamide (PA) or the polymer composition (C), wherein the article is a cookware component.

### DETAILED DESCRIPTION OF THE INVENTION

Described herein are amorphous aliphatic polyamides (PA) formed from the polycondensation of monomers in a reaction mixture (RM) including a dicarboxylic acid component (DC) and a diamine component (DA). The dicarboxylic acid component (DC) includes 25 mol % to 80 mol % of 1,4-cyclohexane dicarboxylic acid (“1,4-CHDA”) and 20 mol % to 75 mol % of a linear, aliphatic dicarboxylic acid selected from azelaic acid, sebacic acid or a combination thereof. The diamine component (DA) includes a cycloaliphatic diamine selected from the group consisting of isophoronediamine (“IPDA”); 4,4′-methylenebis(2-methylcyclohexylamine) (“MACM”); 4,4′-methylene-bis-cyclohexane (“PACM”); 1,3-bis(aminomethyl)cyclohexane (“1,3-BAC”), bis(aminomethyl)norborane (“BAMN”) and any combination of two or more thereof. It was surprisingly found that the polyamides (PA), while being biobased (at least in part due to the linear aliphatic dicarboxylic acid), had excellent optical clarity, improved UV stability and desirably high Tg, relative to amorphous polyamides known in the industry that are prepared from cycloaliphatic diamine, aromatic diacid and aliphatic linear long chain diacid containing at least 12 carbon atoms. As used herein, the term aliphatic also includes reference to cycloaliphatic, unless explicitly indicated otherwise.

The polyamides (PA) have a relatively high glass transition temperature (“Tg”), such that they can be desirably incorporated into application settings in which the polyamide (PA) is exposed to elevated temperatures, as described in more detail below. The polyamides (PA) have a Tg of from 160° C. to 260° C. The relatively high Tg is due, at least in part, to the cyclic aliphatic dicarboxylic acid in the dicarboxylic acid component (DC), as well as to the lack of amino acids and lactams in the reaction mixture (RM). With respect to the linear aliphatic dicarboxylic acid, not only does it provide biobased content to the polyamide (PA), but also mitigates against decreased Tg before and/or after conditioning in humid atmosphere. That is, where the linear aliphatic dicarboxylic acid has less than 9 carbon atoms or greater than 10 carbon atoms, the Tg the of resulting polyamide before and/or after conditioning in humid atmosphere is reduced, relative to a corresponding reaction mixture (RM) in which the dicarboxylic acid component (DC) contains a linear, aliphatic dicarboxylic acid having 9 or 10 carbon atoms. Similarly, polyamides including recurring units formed from the polycondensation of reaction mixtures including amino acids, lactams such as caprolactam or tertiary amines generally have increased moisture absorption, relative to analogous polyamides formed from reaction mixtures (RM) free of such monomers. Therefore, when present in application settings exposed to the ambient environment, the increased moisture absorption results in a decreased Tg. Accordingly, the reaction mixture (RM) is free of amino acids, lactams and tertiary amines. As used herein and unless explicitly stated otherwise, “free of” means that concentration of the indicated monomer is less than 5 mol %, less than 3 mol %, less than 2 mol %, less than 1 mol %, less than 0.5 mol % or less than 0.1 mol %, relative to the total number of moles of components in the reaction mixture (RM), dicarboxylic acid component (DC) or diamine component, as the case may be, that undergo polycondensation to form the polyamide (PA). For example, free of a lactam means the concentration of the lactam is within the ranges above, with respect to the total number of moles of monomers in the reaction mixture (RM). As another example, free of an indicated dicarboxylic acid means the concentration of the indicated dicarboxylic acid is within the ranges given above, relative to the total number of moles of dicarboxylic acids in the dicarboxylic acid component (DC). As yet another example, free of an indicated diamine means the concentration of the indicated diamine is within the ranges given above, relative to the total number of moles of diamines in the diamine component (DA).

Azelaic acid and sebacic acid impart significant biobased content to the polyamide (PA). In particular, azelaic acid can be produced from the ozonolysis of oleic acid, which is a naturally occurring fatty acid present in animal and vegetable fats and oils. Sebacic acid is also produced from biological sources. In particular, sebacic acid is produced from castor oil. Accordingly, not only do the polyamides (PA) have a significant biobased content, but that biobased content additionally promotes elevated Tgs in the polyamides (PA), as described in detail above. Biobased content (also referred to herein as biobased carbon content) can be calculated as in the Examples In some embodiments, the polyamides (PA) have a biobased carbon content of at least 15% or at least 20%.

Because of the combination of properties, notably because they are amorphous and transparent (e.g. have a high optical clarity), the polyamides (PA) are suitable in applications such as cookware windows or containers where a good resistance to hot water/steam is necessary while being able to see the ingredients through.

The polyamides (PA) of this invention are aliphatic (cycloaliphatic in particular), which are particularly important to ensure UV resistance and limit side reactions such as branching. That is, the reaction mixture (RM) is free of aromatic monomers (monomers containing an aryl group) that polycondense to form recurring units in the polyamide (PA). That is, the concentration of aromatic monomers that polycondense to form recurring units in the polyamide (PA) is less than 5 mol %, less than 2 mol %, less than 1 mol % or less than 0.5 mol %, relative to the total number of monomers in the reaction mixture (RM) that polycondense to form the polyamide (PA). In some such embodiments, the concentration of aromatic dicarboxylic acid monomers and the concentration of aromatic diamine monomers in the respective dicarboxylic acid component (DC) and diamine component (DA), respectively, are less than 5 mol %, less than 2 mol %, less than 1 mol % or less than 0.5 mol %.

According to embodiment, the recurring units of the polyamide (PA) are substantially free of any aromatic moiety (e.g. aryl ring or benzene ring), more particularly do not contain any aromatic moiety.

In some embodiments, the reaction mixture (RM) consists essentially of or consists of the dicarboxylic acid component (DC) and the diamine (DA) component, as described above. In such embodiments, the total concentration of dicarboxylic acids and diamines in the reaction mixture (RM) is at least 95 mol %, at least 97 mol %, at least 98 mol %, at least 99 mol % or at least 99.5 mol %, relative to the total number of moles of monomers in the reaction mixture (RM) that polycondense to form the polyamide (PA).

In the present application, any description, even though described in relation to a specific embodiment, is applicable to and interchangeable with other embodiments of the present disclosure. Where an element or component is said to be included in and/or selected from a list of recited elements or components, it should be understood that in related embodiments explicitly contemplated here, the element or component can also be any one of the individual recited elements or components, or can also be selected from a group consisting of any two or more of the explicitly listed elements or components; any element or component recited in a list of elements or components may be omitted from such list; and any recitation herein of numerical ranges by endpoints includes all numbers subsumed within the recited ranges as well as the endpoints of the range and equivalents.

### The Dicarboxylic Acid Component (DC)

The dicarboxylic acid component (DC) component includes all dicarboxylic acids in the reaction mixture (RM), including 25 mol % to 80 mol % of 1,4-CHDA and 20 mol % to 75 mol % of a linear, aliphatic dicarboxylic acid selected from azelaic acid, sebacic acid or a combination thereof. When referring to the concentration of dicarboxylic acids, it will be understood that the concentration is relative to the total number of moles of all dicarboxylic acids in the dicarboxylic acid component (DC), unless explicitly noted otherwise.

### Concentration of 1,4-CHDA

In some embodiments, the 1,4-CHDA concentration is from 25 mol % to 80 mol % or from 30 mol % to 80 mol %. In some embodiments, the 1,4-CHDA concentration is from 25 mol % to 75 mol % or from 30 mol % to 75 mol %. In some embodiments, the 1,4-CHDA concentration is from 25 mol % to 60 mol % or from 30 mol % to 60 mol %. In some embodiments, the 1,4-CHDA concentration is from 25.0 mol % to 65.0 mol %.

In some embodiments, the 1,4-CHDA concentration is from 45.0 mol % to 65.0 mol %. In this range of concentration, the Tg may be at least 185° C. or even at least 195° C. The 1.4-CHDA concentration may be as defined in claim 17.

A skilled person understands that these concentrations given for 1,4-CHDA correspond also to the proportions of recurring units R_{PA1} in the polyamide (PA). The proportion of recurring units R_{PA1} in the polyamide (PA) may therefore be as defined in claim 18.

### Concentration of Linear, Aliphatic Dicarboxylic Acid

In some embodiments, the linear, aliphatic dicarboxylic acid concentration is from 20 mol % to 75 mol % or from 30 mol % to 75 mol %. In some embodiments, the linear, aliphatic dicarboxylic acid concentration is from 25 mol % to 60 mol % or from 30 mol % to 60 mol %. In some embodiments, the linear, aliphatic dicarboxylic acid concentration is from 35.0 mol % to 75.0 mol %. The person of ordinary skill in the art will recognized that additional linear, aliphatic dicarboxylic acid concentrations ranges within the explicitly stated ranges above are contemplated and within the present disclosure.

A skilled person understands that these concentrations for the linear, aliphatic dicarboxylic acid correspond also to the proportions of recurring units R_{PA2} in the polyamide (PA).

In some embodiments, the dicarboxylic acid component (DC) consists essentially of or consist of the 1,4-CHDA and the linear, aliphatic dicarboxylic acid. In such embodiments, the total concentration of the 1,4-CHDA and linear, aliphatic dicarboxylic acid is at least 95 mol %, at least 97 mol %, at least 98 mol %, at least 99 mol % or at least 99.5 mol %.

The invention thus more particularly relates to a polyamide (PA), the recurring units of which consist essentially of or consist of the recurring units R_{PA1} and R_{PA2}.

### The Diamine Component (DA)

The Diamine Component (DA) includes all diamines in the reaction mixture (RM), including a cycloaliphatic diamine selected from the group consisting of IPDA, MACM, PACM, 1,3-BAC, BAMN, 1,2-cyclohexane diamine, 1,3-cyclohexane diamine, 1,4-cyclohexane diamine, 4-methylcyclohexane-1,3-diamine, 2-methylcyclohexane-1,3-diamine, 1,8-diamino-p-menthane, 2,2-bis(4-aminocyclohexyl)propane (“PACP”), 4,4′-methanediylbis(2,6-dimethylcyclohexanamine) and any combination of two or more thereof. Preferably, the cycloaliphatic diamine is selected from the group consisting of IPDA, MACM, PACM, 1,3-BAC, BAMN and any combination of two or more thereof. Most preferably, the cycloaliphatic diamine is selected from the group consisting of IPDA, MACM, PACM or any combination of two or more thereof.

According to a preferred embodiment (E), the diamine component (DA) includes IPDA. According to this preferred embodiment (E), the diamine is IPDA or a combination of IPDA and at least one diamine selected in the group consisting of MACM, PACM, 1,3-BAC and BAMN or a combination of IPDA and at least one diamine selected in the group consisting of MACM and PACM. Even more particularly, the diamine is IPDA or a combination of IPDA and MACM or a combination of IPDA, MACM and PACM. According to this embodiment (E), the molar ratio IPDA/diamine(s) other than IPDA in the reaction mixture (RM) may preferably be between 50/50 and 100/0. With this preferred embodiment (E), the polyamide exhibit a nice combination of properties.

As mentioned above, the polyamides (PA) are amorphous. As such, to the extent that the cycloaliphatic diamine in the diamine component (DA) is PACM, the trans-trans isomer ratio is less than 30 mol % of the overall content of isomers (including cis-cis, trans-trans, cis-trans) as measured through Gas chromatography. Where the trans-trans isomer ratio is 30 mol % or greater, the resulting polyamide is generally semi-crystalline. The trans-trans isomer corresponds to the stereoisomer for which cyclohexane substituents (—NH₂ and —CH₂— namely) are placed on opposite face of the cyclohexane ring, for both cyclohexane rings that constitutes the PACM molecule.

In some embodiments, the concentration of the cycloaliphatic diamine is at least 75 mol %, at least 80 mol %, at least 85 mol %, at least 90 mol % at least 95 mol % or at least 99 mol %. In some embodiments, the diamine component (DA) consists essentially of the cycloaliphatic diamine. In such embodiments, the cycloaliphatic diamine concentration is at least 95 mol %, at least 97 mol %, at least 98 mol %, at least 99 mol % or at least 99.5 mol %. When referring to the concentration of diamines, it will be understood that the concentration is relative to the total number of moles of all diamines in the diamine component (DA), unless explicitly noted otherwise.

### The Polyamide (PA) of the Invention

The polyamide (PA) is formed from the polycondensation of the monomers in the diamine component and dicarboxylic acid component, as described above. The polyamide (PA) includes or comprises the recurring units R_{PA1} and R_{PA2}, represented by the following formulae, respectively:

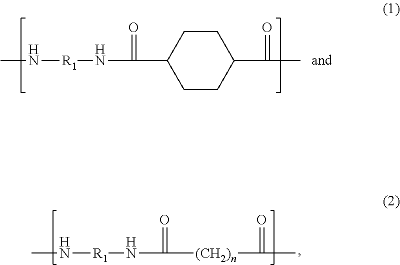

where R₁ is a divalent radical of the diamine selected from the group consisting of IPDA, MACM, PACM, 1,3-BAC and BAMN and n is 7 or 8. For clarity, the divalent radical of the diamine is the divalent radical formed from removing the two amines of the indicated diamine. Additionally n is 7 when the dicarboxylic acid component (DC) includes azelaic acid and n is 8 when the dicarboxylic acid component (DC) includes sebacic acid.

As mentioned previously, the aliphatic dicarboxylic acid is selected from the group consisting of azelaic acid (HOOC—(CH₂)₇—COOH), sebacic acid (HOOC—(CH₂)₈—COOH) and a combination thereof. Therefore, in the formulae above, n may be 7. n may also be 8. It is also possible that the polyamide (PA) contains the recurring units with n=7 and the recurring units with n=8.

According to the preferred embodiment (E), the recurring units R_{PA1} and R_{PA2} include the divalent radical of IPDA. According to this preferred embodiment (E), R1 is the divalent radical of IPDA or of a combination of IPDA and at least one diamine selected in the group consisting of MACM, PACM, 1,3-BAC and BAMN or of a combination of IPDA and at least one diamine selected in the group consisting of MACM and PACM. Even more particularly, R1 is the divalent radical of IPDA or of a combination of IPDA and MACM or of a combination of IPDA, MACM and PACM. According to embodiment (E), the molar ratio divalent radical of IPDA/divalent radical of the diamine(s) other than IPDA may preferably be between 50/50 and 100/0.

In some embodiments, the total concentration of recurring units R_{PA1} and R_{PA2} in the polyamide (PA) is at least 50 mol %, at least 60 mol %, at least 70 mol %, at least 80 mol %, at least 90 mol %, at least 95 mol %, at least 97 mol %, at least 98 mol %, at least 99 mol % or at least 99.5 mol %. When referring to mol % of a recurring unit, it will be understood that the concentration is relative to the total number of recurring units in the indicated polymer, unless explicitly noted otherwise. The invention also more particularly relates to a polyamide (PA), the recurring units of which consist essentially of or consist of the recurring units R_{PA1} and R_{PA2}.

In some embodiments, the ratio of the number of recurring units R_{PA1} to R_{PA2} is from 2.3 to 0.4.

As explained in detail above, the polyamides (PA) have a relatively high Tg. More specifically, the polyamides (PA) have a Tg of from 160° C. to 260° C., preferably 160° C. to 220° C. Tg may at least 185° C., even at least 195° C. Tg can be measured according to ASTM D3418. Tg can be measured by DSC (Differential Scanning Calorimetry), notably by DSC measurements between 40° C. and 320° C. The method disclosed in the experimental section can be used. The method consists in evaluating Tg by DSC between 40° C. and 320° C. wherein after a 1^{st} heating ramp, 1^{st} cooling and second heating (all at 10° C./min), the Tg is measured at the 3^{rd} heating (40° C./min).

Additionally, as explained above, the polyamides (PA) are amorphous. As used herein, an amorphous polyamide has a heat of fusion (“ΔH_{f}”) of less than 5 Joules per gram (“J/g”), preferably less than 3 J/g, more preferably less than 2 J/g, most preferably less than 1 J/g. ΔH_{f} can be measured according to ASTM D3418 using a heating rate of 20° C./minute.

In some embodiments, the polyamides (PA) have a number average molecular weight (“Mn”) ranging from 1,000 g/mol to 40,000 g/mol, for example from 2,000 g/mol to 35,000 g/mol, from 4,000 to 30,000 g/mol, or from 5,000 g/mol to 20,000 g/mol. The number average molecular weight Mn can be determined by gel permeation chromatography (GPC) using ASTM D5296 with PMMA standards.

### Process of Preparation of the Polyamides (PA)

The polyamide (PA) described herein can be prepared by any conventional method adapted to the synthesis of polyamides and polyphthalamides. A convenient route for the preparation of the polyamide (PA) is polycondensation. This involves heating the reaction mixture (RM) at a temperature T* which is a least higher than Tg+50° C.

Preferentially, the polyamide (PA) of the invention is prepared by reacting (by heating) the monomers in presence of less than 30 wt. % of water, preferentially less than 20 wt. %, even more preferentially less than 10 wt. %, up to a temperature T* of at least Tg+50° C., where wt. % is relative to the total weight of the reaction mixture.

T* is preferably at least 250° C., even at least 275° C.

The polycondensation is advantageously performed in a well stirred vessel such as a stirred reactor, an extruder or a kneader. The polycondensation is preferably performed in a stirred reactor. The vessel is also advantageously equipped with means to remove the volatile products of the reaction. As the viscosity of the reaction mixture increases over time, the stirrer is adapted to provide sufficient stirring to the reaction mixture at the beginning of the polymerization and when the conversion of the polycondensation is nearly complete.

The conditions disclosed in example 1 may conveniently be used for the preparation of the polyamide of the invention.

In some embodiments, the total number of moles of diamine(s) in the reaction mixture is substantially equimolar to the total number of moles of dicarboxylic acids in the reaction mixture. As used herein, substantial equimolar denotes a value that is ±15% of the indicated number of moles. For example, in the context of the diamine and dicarboxylic acid concentrations in the reaction mixture, total number of moles of diamines in the reaction mixture is ±15% of the total number of moles of dicarboxylic acids in the reaction mixture. The polyamides (PA) may contain a chain limiter, which is a monofunctional molecule capable of reacting with the amine or carboxylic acid moiety, and is used to control the molecular weight of the polyamide (PA). For example, the chain limiter (which is added to the reaction mixture) can be acetic acid, propionic acid, benzoic acid and/or benzylamine.

A catalyst can also conveniently be used. Examples of catalyst are phosphorous acid, ortho-phosphoric acid, meta-phosphoric acid, alkali-metal hypophosphite such as sodium hypophosphite and phenylphosphinic acid. A stabilizer, such as a phosphite, may also be used.

### Polymer Composition (C)

The polymer composition (C) comprises the polyamide (PA) of the present invention. The polyamide (PA) may be present in the composition (C) in a total amount of greater than 30 wt. %, greater than 35 wt. %, greater than 40 wt. % or greater than 45 wt. %, based on the total weight of the polymer composition (C). This amount may be greater than 50.0 wt % or even greater than 60.0 wt %.

The polyamide (PA) may be present in the composition (C) in a total amount of less than 99.95 wt. %, less than 99 wt. %, less than 95 wt. %, less than 90 wt. %, less than 80 wt. %, less than 70 wt. % or less than 60 wt. %, based on the total weight of the polymer composition (C). The polyamide (PA) may for example be present in the composition (C) in an amount ranging between 35 and 70 wt. %, for example between 40 and 55 wt. %, based on the total weight of the polymer composition (C).

The composition (C) may also comprise a component selected from the group consisting of reinforcing agents, tougheners, plasticizers, colorants, pigments, antistatic agents, dyes, lubricants, thermal stabilizers, light stabilizers, flame retardants, nucleating agents, antioxidants, processing aids and any combination of two or more thereof.

A large selection of reinforcing agents, also called reinforcing fibers or fillers, may be added to the composition according to the present invention. They can be selected from fibrous and particulate reinforcing agents. A fibrous reinforcing filler is considered herein to be a material having length, width and thickness, wherein the average length is significantly larger than both the width and thickness. Generally, such a material has an aspect ratio, defined as the average ratio between the length and the largest of the width and thickness of at least 5, at least 10, at least 20 or at least 50. In some embodiments, the reinforcing fibers (e.g. glass fibers or carbon fibers) have an average length of from 3 mm to 50 mm. In some such embodiments, the reinforcing fibers have an average length of from 3 mm to 10 mm, from 3 mm to 8 mm, from 3 mm to 6 mm, or from 3 mm to 5 mm. In alternative embodiments, the reinforcing fibers have an average length of from 10 mm to 50 mm, from 10 mm to 45 mm, from 10 mm to 35 mm, from 10 mm to 30 mm, from 10 mm to 25 mm or from 15 mm to 25 mm. The average length of the reinforcing fibers can be taken as the average length of the reinforcing fibers prior to incorporation into the polymer composition (C) or can be taken as the average length of the reinforcing fiber in the polymer composition (C).

The reinforcing filler may be selected from mineral fillers (such as talc, mica, kaolin, calcium carbonate, calcium silicate, magnesium carbonate), glass fibers, carbon fibers, synthetic polymeric fibers, aramid fibers, aluminum fibers, titanium fibers, magnesium fibers, boron carbide fibers, rock wool fibers, steel fibers and wollastonite.

Among fibrous fillers, glass fibers are preferred; they include chopped strand A-, E-, C-, D-, S- and R-glass fibers, as described in chapter 5.2.3, p. 43-48 of Additives for Plastics Handbook, 2^{nd} edition, John Murphy. Preferably, the filler is chosen from fibrous fillers. It is more preferably a reinforcing fiber that is able to withstand the high temperature applications.

The reinforcing agents may be present in the composition (C) in a total amount of greater than 15 wt. %, greater than 20 wt. % by weight, greater than 25 wt. % or greater than 30 wt. %, based on the total weight of the polymer composition (C. The reinforcing agents may be present in the composition (C) in a total amount of less than 65 wt. %, less than 60 wt. %, less than 55 wt. % or less than 50 wt. %, based on the total weight of the polymer composition (C).

The reinforcing filler may for example be present in the composition (C) in an amount ranging between 20 and 60 wt. %, for example between 30 and 50 wt. %, based on the total weight of the polymer composition (C).

The composition (C) may also comprise a toughener. A toughener exhibits generally a low Tg, with a Tg for example below room temperature, below 0° C. or even below −25° C. As a result of their low Tg, the tougheners are typically elastomeric at room temperature. Tougheners can be functionalized polymer backbones.

The polymer backbone of the toughener can be selected from elastomeric backbones comprising polyethylenes and copolymers thereof, e.g. ethylene-butene; ethylene-octene; polypropylenes and copolymers thereof, polybutenes; polyisoprenes; ethylene-propylene-rubbers (EPR); ethylene-propylene-diene monomer rubbers (EPDM); ethylene-acrylate rubbers; butadiene-acrylonitrile rubbers, ethylene-acrylic acid (EAA), ethylene-vinylacetate (EVA); acrylonitrile-butadiene-styrene rubbers (ABS), block copolymers styrene ethylene butadiene styrene (SEBS); block copolymers styrene butadiene styrene (SBS); core-shell elastomers of methacrylate-butadiene-styrene (MBS) type, or mixture of one or more of the above.

When the toughener is functionalized, the functionalization of the backbone can result from the copolymerization of monomers which include the functionalization or from the grafting of the polymer backbone with a further component.

Specific examples of functionalized tougheners are notably terpolymers of ethylene, acrylic ester and glycidyl methacrylate, copolymers of ethylene and butyl ester acrylate; copolymers of ethylene, butyl ester acrylate and glycidyl methacrylate; ethylene-maleic anhydride copolymers; EPR grafted with maleic anhydride; styrene copolymers grafted with maleic anhydride; SEBS copolymers grafted with maleic anhydride; styrene-acrylonitrile copolymers grafted with maleic anhydride; ABS copolymers grafted with maleic anhydride.

The toughener may be present in the composition (C) in a total amount of greater than 1 wt. %, greater than 2 wt. % or greater than 3 wt. %, based on the total weight of the composition (C). The toughener may be present in the composition (C) in a total amount of less than 30 wt. %, less than 20 wt. %, less than 15 wt. % or less than 10 wt. %, based on the total weight of the polymer composition (C).

The polymer composition (C) may also comprise other conventional additives commonly used in the art, including plasticizers, colorants, pigments (e.g. black pigments such as carbon black and nigrosine), antistatic agents, dyes, lubricants (e.g. linear low density polyethylene, calcium or magnesium stearate or sodium montanate), thermal stabilizers, light stabilizers, flame retardants (e.g. halogen free flame retardants), nucleating agents and antioxidants. Examples of halogen free flame retardants include, but are not limited to, phosphinic acid salts of aluminium.

The composition (C) may comprise at least one halogen-free flame retardant, notably a phosphorous-based flame retardant. The phosphorous-based flame retardant may be selected in the group consisting of metal alkyl phosphinates. An example of metal alkyl phosphinate is aluminum diethyl phosphinate, for instance the one known under the trade name Exolit® from Clariant. Another example of phosphorous-based flame retardant which ensures a good level of flame retardancy is disclosed below:

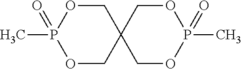

The polymer composition (C) may also comprise one or more other polymers, preferably polyamides different from the polyamide (PA) of the present invention. Mention can be made notably of semi-crystalline or amorphous polyamides, such as aliphatic polyamides, semi-aromatic polyamides, and more generally the polyamides obtained by polycondensation between an aromatic or aliphatic saturated diacid and an aliphatic saturated or aromatic primary diamine, a lactam, an amino-acid or a mixture of these different monomers.

### Preparation of the Polymer Composition (C)

The invention further pertains to a method of making the composition (C) as above detailed, said method comprising melt-blending the polyamide (PA) and the specific components, e.g. a filler, a toughener, a stabilizer, and of any other optional additives.

Any melt-blending method may be used for mixing polymeric ingredients and non-polymeric ingredients in the context of the present invention. For example, polymeric ingredients and non-polymeric ingredients may be fed into a melt mixer, such as single screw extruder or twin screw extruder, agitator, single screw or twin screw kneader, or Banbury mixer, and the addition step may be addition of all ingredients at once or gradual addition in batches. When the polymeric ingredient and non-polymeric ingredient are gradually added in batches, a part of the polymeric ingredients and/or non-polymeric ingredients is first added, and then is melt-mixed with the remaining polymeric ingredients and non-polymeric ingredients that are subsequently added, until an adequately mixed composition is obtained. If a reinforcing agent presents a long physical shape (for example, long fibers as well as continuous fibers), drawing extrusion or pultrusion may be used to prepare a reinforced composition.

### Articles and Applications

The present invention also relates to articles comprising the polyamide (PA) of the present invention and to articles comprising the copolymer composition (C) described above. The articles can be desirably incorporated into healthcare applications including, but not limited to, sutures, hemodialysis membranes, vascular catheters, scaffolds (e.g., for ligament and tendon repair), catheter balloons, wound dressings, breathing masks (e.g. face shield component), and medical tubing.

The articles can also be desirably incorporated into cookware components including, but not limited to, coffee machines components, catering containers, vacuum containers, and kitchen utensils.

The article can notably be used in mobile electronics, LED packaging, oil and gas components, food contact components (including, but not limited to, food film and casing, baby bottle, bottle for drinking water), electrical and electronic components (including, but not limited to, power unit components for computing, data-system and office equipment and surface mounted technology compatible connectors and contacts), medical device components (including but not limiting to components for hearing aids and earphones, observation window for protective masks, consumer components (household appliances, optics spectacle frames for prescription glasses and sunglasses, frames for safety glasses, sun protective lenses/spectacle lenses, cosmetic packaging, toothbrushes) construction components (including, but not limited to, pipes, connectors, manifolds and valves, for cooling and heating systems; boiler and meter components; gas systems pipes and fittings); and electrical protection devices for mini-circuit breakers, contactors, switches and sockets), industrial components, plumbing components (including, but not limited to, pipes, valves, fittings, manifolds, shower taps and shower valves), automotive components, and aerospace components (including, but not limited to, interior cabin components).

The article can, for example, be a mobile electronic device component. As used herein, a “mobile electronic device” refers to an electronic device that is intended to be conveniently transported and used in various locations. A mobile electronic device can include, but is not limited to, a mobile phone, a personal digital assistant (“PDA”), a laptop computer, a tablet computer, a wearable computing device (e.g., a smart watch, smart glasses and the like), a camera, a portable audio player, a portable radio, global position system receivers, and portable game consoles.

The mobile electronic device component may, for example, comprise a radio antenna and the composition (C). In this case, the radio antenna can be a WiFi antenna or an RFID antenna. The mobile electronic device component may also be an antenna housing. Further examples of mobile electronic device components include, but are not limited to, microspeakers, microswitches, microreceivers, connectors, cameras modules, camera lens protectors, back housings, battery covers, chassis and frames.

In some embodiments, the mobile electronic device component is an antenna housing. In some such embodiments, at least a portion of the radio antenna is disposed on the polymer composition (C). Additionally or alternatively, at least a portion of the radio antenna can be displaced away (e.g. not contacting) from the polymer composition (C). In some embodiments, the device component can be of a mounting component with mounting holes or other fastening device, including but not limited to, a snap fit connector between itself and another component of the mobile electronic device, including but not limited to, a circuit board, a microphone, a speaker, a display, a battery, a cover, a housing, an electrical or electronic connector, a hinge, a radio antenna, a switch, or a switchpad. In some embodiments, the mobile electronic device can be at least a portion of an input device.

Examples of oil and gas components include, but are not limited to, compressor rings, poppets, back-up seal rings, electrical connectors, labyrinth seals, motor end plates, bearings, bushings, suck rod guides and down hole tubing.

Examples of automotive components include, but are not limited to, components in thermal management systems (including, but not limited to, thermostat housings, water inlet/outlet valves, water pumps, water pump impellers, and heater cores and end caps), air management system components (including, but not limited to, turbocharger actuators, turbocharger by-pass valves, turbocharger hoses, EGR valves, CAC housings, exhaust gas recirculation systems, electronic controlled throttle valves, and hot air ducts), transmission components and launch device components (including, but not limited to, dual clutch transmissions, automated manual transmissions, continuously variable transmissions, automatic transmissions, torque convertors, dual mass flywheels, power takeoffs, clutch cylinders, seal rings, thrust washers, thrust bearings, needle bearings, and check balls), automotive electronic components, automotive lighting components (including, but not limited to, motor end caps, sensors, ECU housings, bobbins and solenoids, connectors, circuit protection/relays, actuator housings, Li-Ion battery systems, and fuse boxes), traction motor and power electronic components (including, but not limited to, battery packs), fuel and selective catalytic reduction (“SCR”) systems (including, but not limited to, SCR module housings and connectors, SCR module housings and connectors, fuel flanges, rollover valves, quick connects, filter housings, fuel rails, fuel delivery modules, fuel hoses, fuel pumps, fuel injector o-rings, and fuel hoses), fluid system components (e.g. fuels system components) (including, but not limited to inlet and outlet valves and fluid pump components), interior components (e.g. dashboard components, display components, and seating components), and structural and lightweighting components (e.g. gears and bearings, sunroofs, brackets and mounts, electrical battery housings, thermal management components, braking system elements, and pump and EGR systems).

The polyamide (PA), polymer composition (C) and article prepared therefrom may also be used as a gas barrier material for packaging applications, in mono or multilayer articles.

The polyamide (PA), polymer composition (C) and article prepared therefrom can also be used in automotive applications, for example in air induction systems, cooling and heating systems, drivetrain and fuel systems, beams and structural supports, pans and covers.

The article can be molded from the polyamide (PA) or polymer composition (C) of the present invention, by any process adapted to thermoplastics, e.g. extrusion, injection molding, blow molding, rotomolding or compression molding. Polyamide (PA) and polymer composition (C) may also be used in overmolding pre-formed shapes to build hybrid structures. The article can be printed from the polyamide (PA) or polymer composition (C) of the present invention, by a process comprising a step of extrusion of the material, which is for example in the form of a filament, or comprising a step of laser sintering of the material, which is in this case in the form of a powder.

The present invention also relates to a method for manufacturing a three-dimensional (3D) object with an additive manufacturing system, comprising:
providing a part material comprising the polyamide (PA) or polymer composition (C) of the present invention, and
printing layers of the three-dimensional object from the part material.

The polyamide (PA) or polymer composition (C) can therefore be in the form of a thread or a filament to be used in a process of 3D printing, e.g. Fused Filament Fabrication, also known as Fused Deposition Modelling (FDM).

The polyamide (PA) or polymer composition (C) can also be in the form of a powder, for example a substantially spherical powder, to be used in a process of 3D printing, e.g. Selective Laser Sintering (SLS).

The polyamide (PA) or polymer composition (C) can also be desirably incorporated into thermoplastic composites (e.g. tapes) with continuous glass or carbon fibers.

### Use of the Polyamides (PA), Composition (C) and Articles

The present invention relates to the use of the above-described polyamides (PA), composition (C) or articles for manufacturing a mobile electronic device component, as described above.

The present invention also relates to the use of the above-described polyamides (PA) or composition (C) for 3D printing an object.

Should the disclosure of any patents, patent applications, and publications which are incorporated herein by reference conflict with the description of the present application to the extent that it may render a term unclear, the present description shall take precedence.

### EXPERIMENTAL SECTION

The present examples demonstrate the synthesis, thermal performance, and mechanical performance of the polyamides. The raw materials used to form the samples as provided below:
IPDA (obtained from Aldrich)
1,4-CHDA (obtained from Aldrich)
1,6-hexanedioic acid (obtained from Aldrich) (“C6” in Table 1), also known as adipic acid.
1,10-decanedioic acid (obtained from Aldrich) (“C10” in Table 1), also known as sebacic acid. This monomer has 10 biobased Carbon atoms.
1,9-nonanedioic acid (obtained from Aldrich) (“C9” in Table 1), also known as azelaic acid. This monomer has 9 biobased Carbon atoms.
1,12-dodecanedioic acid (obtained from Aldrich)
1,15-pentadecanedioic acid (obtained from Cathay) (“C15” in Table 1).
MACM (obtained from Aldrich)
PACM (obtained from Acros)

### Synthesis of Polyamides

This example demonstrates the synthesis of the polyamides. The sample parameters for each reaction mixture are provided in Table 1. The biobased carbon content in a polymer is defined as the ratio of the number of biobased carbon atoms (coming from biobased monomers) in the polymer over all Carbon atoms in the polymer (coming from biobased and non-biobased monomers).

TABLE 1

| | | Dicarboxylic | | | Biobased | |
|---|---|---|---|---|---|---|
| | Diamine | Acid | | | Carbon | |
| | Component | Component | Amorphous | Tg | content | Transparency |
| Ex. | (mol %) | (mol %) | (YES/NO) | (° C.) | (%) | (YES/NO) |
| E1 | IPDA (100) | C10 (70) | YES | 170 | 36 | YES |
| | | CHDA (30) | | | | |
| E2 | IPDA (100) | C10 (40) | YES | 204 | 21 | YES |
| | | CHDA (60) | | | | |
| CE1 | IPDA (100) | CHDA (100) | YES | 242 | 0 | YES |
| CE2 | IPDA (100) | C10 (100) | YES | 131 | 53 | YES |
| E3 | IPDA (50) | C10 (50) | YES | 196 | 23 | YES |
| | MACM | CHDA (50) | | | | |
| | (50) | | | | | |
| E4 | MACM | C9 (70) | YES | 187 | 27 | YES |
| | (100) | CHDA (30) | | | | |
| CE3 | MACM | C9 (100) | YES | 145 | 38 | YES |
| | (100) | | | | | |
| CE4 | MACM | CHDA (100) | NO | N.M. | 0 | NO |
| | (100) | | | | | |
| CE5 | MACM | CHDA (70) | NO | N.M. | 0 | NO |
| | (100) | C6 (30) | | | | |
| CE6 | MACM | C12 (70) | YES | 176 | 0 | YES |
| | (100) | CHDA (30) | | | | |
| CE7 | MACM | C15 (70) | YES | 162 | 0 | YES |
| | (100) | CHDA (30) | | | | |
| E5 | IPDA (50) | CHDA (50) | YES | 188 | 24 | YES |
| | MACM | C10 (50) | | | | |
| | (25) | | | | | |
| | PACM (25) | | | | | |

### Example 1

105.7 g (0.62 mol) of IPDA, 86.4 g (0.43 mol) of 1,10-decanedioic acid, 31.5 g (0.18 mol) of 1,4-CHIDA, 4.1 g of an aqueous solution of sodium hypophosphite monohydrate (5% wt, 2 mmol) and 30 g of deionized water were introduced in a stainless-steel autoclave equipped with a mechanical stirrer. The autoclave was purged with nitrogen, then sealed and the temperature in the reactor was gradually increased up to 300′C. At the same time, the pressure increased and was allowed to reach a maximum of 11 bar, above which the excess vapor was distilled off. The pressure was then released and the autoclave was put under vacuum (˜100 mbar) during 20 min. The resulting polymer was then discharged as a strand and pelletized.

Examples 2, 3, 4, 5, and all Comparative Examples CE1, CE2, CE3, CE4, CE5, CE6 and CE7 are prepared using the same procedure as Example 3 but using the adequate monomer combinations and molar concentrations provided in Table 1.

### Thermal Performance

To demonstrate thermal performance, Tg was measured as described above. More specifically, for each sample, Tg was evaluated through DSC measurements between 40° C. and 320° C. After a 1^{st} heating ramp, 1^{st} cooling and second heating (all at 10° C./min), Tg was measured at 3^{rd} heating (40° C./min). Tg is displayed in Table 1.

Comparison of E1 and E2 with CE1 and CE2, demonstrates that, surprisingly, polyamides formed from a combination of at least a cycloaliphatic diamine (IPDA), CHDA and C10 (1,10-decanedioic acid) diacid resulted in a Tg is higher than 160° C., while simultaneously having a biobased carbon content of at least 20%. Similarly, comparison of E4 with CE3, CE4, CE5, CE6, and CE7, demonstrates, surprisingly, polyamides formed from a combination of at least a cycloaliphatic diamine (MACM), CHDA and a C9 diacid (1,9-nonanedioic acid) resited in an amorphous and transparent polyamide having a Tg>160° C. and a biobased Carbon content of at least 20%.

Furthermore, E4 and E5 demonstrate that, surprisingly, polyamides formed from a combination of at least a cycloaliphatic diamine, CHDA and a C10 diacid (1,10-decanedioic acid), the resulting polyamide was amorphous and transparent polymer, while having a Tg>180° C. and a biobased carbon content of at least 20%.

The embodiments above are intended to be illustrative and not limiting. Additional embodiments are within the inventive concepts. In addition, although the present invention is described with reference to particular embodiments, those skilled in the art will recognized that changes can be made in form and detail without departing from the spirit and scope of the invention. Any incorporation by reference of documents above is limited such that no subject matter is incorporated that is contrary to the explicit disclosure herein.

1. An amorphous polyamide (PA) comprising the recurring units R_{PA1} and R_{PA2} which are represented by the following formulae, respectively, 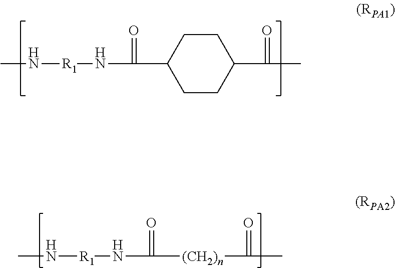
with the following proportions:
from 25.0 to 80.0 mol. % of R_{PA1};
from 20.0 to 75.0 mol. % of R_{PA2};
where:
R₁ is a divalent radical of a diamine selected from the group consisting of isophoronediamine (“IPDA”); 4,4′-methylenebis(2-methylcyclohexylamine) (“MACM”); 4,4′-methylene-bis-cyclohexylamine (“PACM”); 1,3-bis(aminomethyl)cyclohexane (“1,3-BAC”), bis(aminomethyl)norbornane (“BAMN”), 1,2-cyclohexane diamine, 1,3-cyclohexane diamine, 1,4-cyclohexane diamine, 4-methylcyclohexane-1,3-diamine, 2-methylcyclohexane-1,3-diamine, 1,8-diamino-p-menthane, 2,2-bis(4-aminocyclohexyl)propane (“PACP”), 4,4′-methanediylbis(2,6-dimethylcyclohexanamine) and any combination of two or more thereof, and
n is 7 or 8;
and wherein the polyamide (PA) has a glass transition temperature (“Tg”) of from 160° C. to 260° C.

2. An amorphous, aliphatic polyamide (PA) comprising recurring units formed from the polycondensation of a reaction mixture (RM) comprising:
a dicarboxylic acid component (DC) comprising:
25 mol % to 80 mol % of 1,4-cyclohexane dicarboxylic acid (“1,4-CHDA”) and
20 mol % to 75 mol % of a linear, aliphatic dicarboxylic acid selected from the group consisting of azelaic acid, sebacic acid and a combination thereof,
wherein mol % is relative to the total moles of dicarboxylic acids in the dicarboxylic acid component (DA);
a diamine component (DA) comprising:
a cycloaliphatic diamine selected from the group consisting of isophoronediamine (“IPDA”); 4,4′-methylenebis(2-methylcyclohexylamine) (“MACM”); 4,4′-methylene-bis-cyclohexylamine (“PACM”); 1,3-bis(aminomethyl)cyclohexane (“1,3-BAC”), bis(aminomethyl)norbornane (“BAMN”), 1,2-cyclohexane diamine, 1,3-cyclohexane diamine, 1,4-cyclohexane diamine, 4-methylcyclohexane-1,3-diamine, 2-methylcyclohexane-1,3-diamine, 1,8-diamino-p-menthane, 2,2-bis(4-aminocyclohexyl)propane (“PACP”), 4,4′-methanediylbis(2,6-dimethylcyclohexanamine) and any combination of two or more thereof,
the total number of moles of diamine(s) in the reaction mixture being substantially equimolar to the total number of moles of dicarboxylic acids in the reaction mixture,
wherein the polyamide (PA) has a glass transition temperature (“Tg”) of from 160° C. to 260° C., preferably from 160° C. to 220° C., as measured according to ASTM D3418; and
with the provisos:
the reaction mixture (RM) is free of tertiary amines, lactams and amino acids and
if the cycloaliphatic dicarboxylic acid is PACM, the PACM comprises a trans-trans ratio of no more than 30%.

3. The polyamide (PA) of claim 1, wherein the cycloaliphatic diamine is selected from the group consisting of IPDA, MACM, PACM, 1,3-BAC, BAMN and any combination of two or more thereof.

4. The polyamide (PA) of claim 2, comprising the recurring units R_{PA1} and R_{PA2} which are represented by the following formulae, respectively, 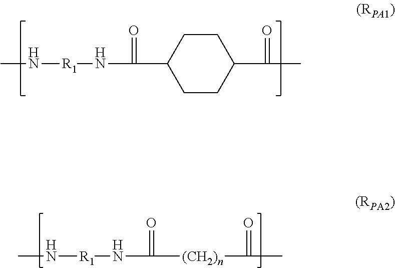
where R₁ is a divalent radical of the diamine, notably selected from the group consisting of IPDA, MACM, PACM, 1,3-BAC, BAMN and any combination of two or more thereof, and n is 7 or 8.

5. The polyamide (PA) of claim 2, wherein the dicarboxylic acid component (DC) consists essentially of the 1,4-CHDA and linear carboxylic acid.

6. The polyamide (PA) of claim 2, wherein the diamine component (DA) consists essentially of the cycloaliphatic diamine.

7. The polyamide (PA) of claim 2, wherein the concentration of the cycloaliphatic diamine is at least 80 mol %.

8. (canceled)

9. (canceled)

10. (canceled)

11. (canceled)

12. (canceled)

13. The polyamide (PA) of claim 2, wherein the diamine is a combination of IPDA and at least one diamine selected in the group consisting of MACM, PACM, 1,3-BAC and BAMN and wherein the molar ratio IPDA/diamine(s) other than IPDA in the reaction mixture (RM) is between 50/50 and 100/0.

14. The polyamide (PA of claim 1, wherein the diamine is a combination of IPDA and at least one diamine selected in the group consisting of MACM, PACM, 1,3-BAC and BAMN and wherein the molar ratio divalent radical of IPDA/divalent radical of the diamine(s) other than IPDA is between 50/50 and 100/0.

15. The polyamide of claim 1 wherein the molar ratio R_{PA1}/R_{PA2} is between 0.4 and 2.3.

16. The polyamide (PA) of claim 2, wherein the concentration of aromatic monomers in the reaction mixture (RM) that polycondense to form the recurring units in the polyamide (PA) is less than 5 mol %, relative to the total number of monomers in the reaction mixture (RM) that polycondense to form the polyamide (PA).

17. The polyamide (PA) of claim 2, wherein the 1,4-CHDA concentration is:
from 25.0 mol % to 80.0 mol %; or
from 30.0 mol % to 80.0 mol %; or
from 25.0 mol % to 75.0 mol %; or
from 30.0 mol % to 75.0 mol %; or
from 25.0 mol % to 60.0 mol %; or
from 30.0 mol % to 60.0 mol %; or
from 25.0 mol % to 65.0 mol %; or
from 45.0 mol % to 65.0 mol %; or
from 25.0 mol. % to 35.0 mol %; or
from 45.0 mol. % to 55.0 mol. %; or
from 55.0 mol % to 65.0 mol %.

18. The polyamide (PA) of claim 1, wherein the proportion of recurring units (R_{PA1}) is:
from 25.0 mol % to 80.0 mol %; or
from 30.0 mol % to 80.0 mol %; or
from 25.0 mol % to 75.0 mol %; or
from 30.0 mol % to 75.0 mol %; or
from 25.0 mol % to 60.0 mol %; or
from 30.0 mol % to 60.0 mol %; or
from 25.0 mol % to 65.0 mol %; or
from 45.0 mol % to 65.0 mol %, or
from 25.0 mol. % to 35.0 mol %; or
from 45.0 mol. % to 55.0 mol. %; or
from 55.0 mol % to 65.0 mol %,
this proportion being based on the total number of recurring units in the polymer.

19. The polyamide (PA) of claim 1, wherein the recurring units of the polyamide (PA) consist essentially of the recurring units R_{PA1} and R_{PA2}.

20. (canceled)

21. The polyamide (PA) of claim 1 exhibiting a heat of fusion (“ΔH_{f}”) of less than 5.0 Joules per gram (“J/g”).

22. The polyamide (PA) of claim 1 having a number average molecular weight (“Mn”) ranging from 1,000 g/mol to 40,000 g/mol, the number average molecular weight Mn being determined by gel permeation chromatography (GPC) using ASTM D5296 with PMMA standards.

23. The polyamide (PA) of claim 1 wherein the recurring units of the polyamide (PA) are substantially free of any aromatic moiety.

24. (canceled)

25. A polymer composition (C) comprising:
the polyamide (PA) of claim 1 and
a component selected from the group consisting of reinforcing agents, tougheners, plasticizers, colorants, pigments, antistatic agents, dyes, lubricants, thermal stabilizers, light stabilizers, flame retardants, nucleating agents, antioxidants, processing aids and any combination of two or more thereof.

26. (canceled)

27. (canceled)

28. (canceled)

29. (canceled)

30. The polymer composition (C) of claim 25, wherein the total amount of polyamide (PA) is in an amount of from:
greater than 30.0 wt. % to less than 99.5 wt
this amount being based on the total weight of the polymer composition (C).

31. (canceled)

32. An article comprising the polyamide (PA) of claim 1, wherein the article is selected from the group consisting of sutures, hemodialysis membranes, vascular catheters, scaffolds, catheter balloons, wound dressings, breathing masks, medical tubing, a filament, a powder, a mobile electronic device component, an automotive component, and a cookware component.

33. (canceled)

34. (canceled)

35. (canceled)

36. (canceled)
